(19) United States
(12) Patent Application Publication
(10) Pub. No.: US 2013/0071917 A1
(43) Pub. Date: Mar. 21, 2013

(54) CAPILLARY FOR APPARATUS OF INSULATED ISOTHERMAL POLYMERASE CHAIN REACTION

(71) Applicants: Chen Su, Taichung City (TW); Ping-Hua Teng, Taichung City (TW); Chien-Chung Jeng, Pingtung County (TW)

(72) Inventors: Chen Su, Taichung City (TW); Ping-Hua Teng, Taichung City (TW); Chien-Chung Jeng, Pingtung County (TW)

(21) Appl. No.: 13/234,655

(22) Filed: Sep. 16, 2011

(51) Int. Cl. C12M 1/40

(52) U.S. Cl. 435/304.1

(57) ABSTRACT

A capillary for apparatus of insulated isothermal polymerase chain reaction is mounted onto a test tube holder. The capillary includes a tube which has an elongated tubular housing space that is cut to form an axial cross section with a distance between left and right edges greater than the distance between front and rear edges. When reactants are filled in the elongated tubular housing space and heated to generate convection, the left and right edges of the axial cross section form a narrower passage to reduce flowing speed of heat flow in the convection and increase cycling duration of the convection. Hence reaction duration of the reactants is prolonged, and reaction efficiency of the apparatus of insulated isothermal polymerase chain reaction also is increased.

### FIELD OF THE INVENTION

The present invention relates to an apparatus for insulated isothermal polymerase chain reaction and particularly to a capillary adopted for an apparatus of insulated isothermal polymerase chain reaction.

### BACKGROUND OF THE INVENTION

Using polymerase chain reaction (PCR) to amplify selective DNA nucleic acid sequence is a well-developed and important medical and bio technology at present. It mainly includes three steps: denaturation, annealing of primers and extension of primers. These three steps require different reaction temperatures. The present PCR reaction requires reactants including template DNA to be amplified, oligonucleotide primer pairs complementary to selected sequence of each strand of the template DNA, thermostable DNA polymerase and deoxynucleoside triphosphate (dNTP). The reactants are repeatedly heated and cooled in the PCR reaction to be circulated at three different temperatures to amplify selected portions of the template DNA nucleic acid sequence.

The first process of denaturation in PCR is to heat the reactants to a high temperature to separate double-strand template DNA into single-strand DNA. A typical denaturation temperature is ranged between 90 and 95° C.

The second process of annealing of primers in PCR is to cool the reactants with the separated single-strand DNA to a lower temperature, so that the primer bind the single-strand DNA to form a complex thereof. A typical annealing temperature is selected according to melting temperature (Tm) of the primer, and usually is ranged between 35 and 65° C.

The third process of extension of primers in PCR is to maintain the reactants at a suitable temperature. Through action of DNA polymerase, the primer is extended to form a new single-strand DNA complementary to each strand of the template DNA. A typical extension temperature is 72° C.

The aforesaid three processes form a cycle and two times of the template DNA can be replicated in each cycle. By repeating the three processes of denaturation, annealing of primers and extension of primers in PCR at three different temperatures cyclically for twenty to forty times, millions of targeted replicates of nucleic acid sequence can be produced.

The conventional PCR machine (i.e. thermal cycler) controls the temperature of reactants via thermal conduction. The reaction container holding the PCR reactants is in contact with a solid metal member with a high thermal conductivity. The metal member is connected to a heating and cooling device, and its temperature is changed via the heating and cooling device. Hence the conventional thermal cycling PCR needs to spend extra time and use additional resources to heat and cool other substances in addition to the PCR reactants. Due to the characteristic of precision of the PCR machine, it is usually very expensive.

Refer to FIGS. 1 and 2 showing another conventional convective polymerase chain reaction (CPCR) technique that costs fewer. It fills conventional CPCR reactants into a circular capillary 1. There are a plurality of capillaries 1 arranged on a test tube holder 2 equipped with a heater (not shown in the drawings) and a cooling device (also not shown in the drawings). The reactants are driven via convection. Through the heater and cooling device, the reactants flow cyclically in the capillary 1. As the capillary 1 has varying temperatures at different regions, when the reactants flow cyclically among the different regions, the three processes of PCR can repeatedly take place in sequence.

As the conventional capillary 1 is formed in a circular shape and arranged in an upright manner and the convection is characterized in a phenomenon of heat flow rising and cool flow falling, the flowing speed in the capillary 1 is fast enough to easily result in mixing of the heat flow and cool flow without obvious separation. This causes decrease of reaction efficiency.

### SUMMARY OF THE INVENTION

Therefore, the primary object of the present invention is to provide a capillary structure to increases cycling duration of convection.

The invention provides a capillary adopted for an apparatus of insulated isothermal polymerase chain reaction that is mounted onto a test tube holder. The capillary includes a tube which has an elongated tubular housing space with an axial direction forming an inclined angle against the plane of the test tube holder. The elongated tubular housing space is cut to form an axial cross section with a distance between left and right edges greater than that between front and rear edges.

Thus, when reactants are filled in the elongated tubular housing space and heated to generate convection, due to the distance between the left and right edges of the axial cross section thereof is greater than that between the front and rear edges, the left and right edges of the axial cross section form a narrower passage, hence can reduce the flowing speed of the heat flow in the convection and increase the cycling duration thereof, i.e. increase the reaction duration of the reactants, thus can increase the reaction efficiency of the apparatus of insulated isothermal polymerase chain reaction.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a perspective view of a conventional capillary.

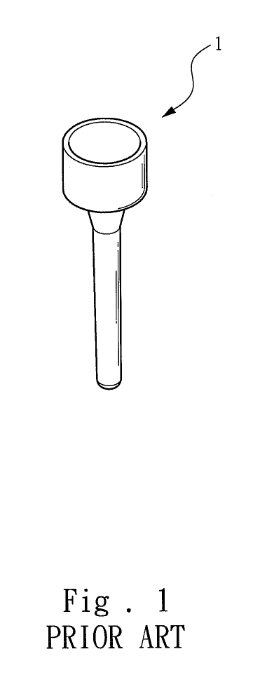

FIG. 2 is a schematic view of a conventional capillary in a holding condition.

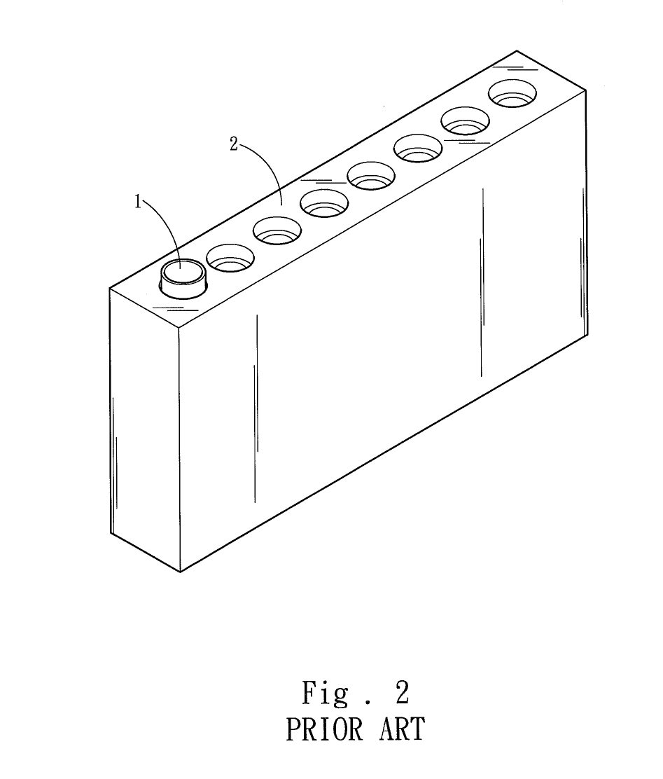

FIG. 3 is a perspective view of a capillary of the invention.

FIG. 4 is a schematic view of the capillary of the invention in a holding condition.

FIG. 5 is a fragmentary sectional view of the capillary of the invention in the holding condition.

FIG. 6 is a schematic view of the capillary of the invention in a use condition.

### DETAILED DESCRIPTION OF THE PREFERRED EMBODIMENT

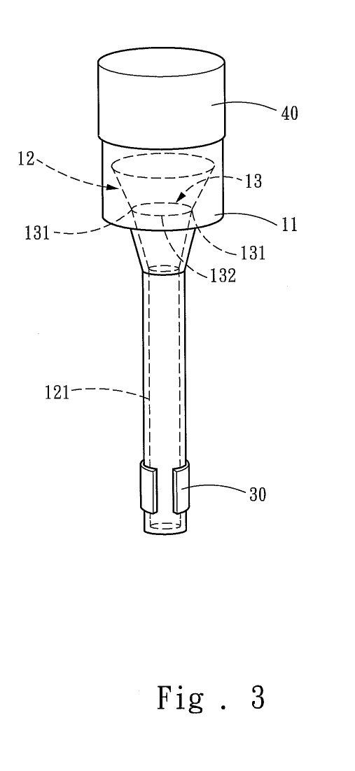

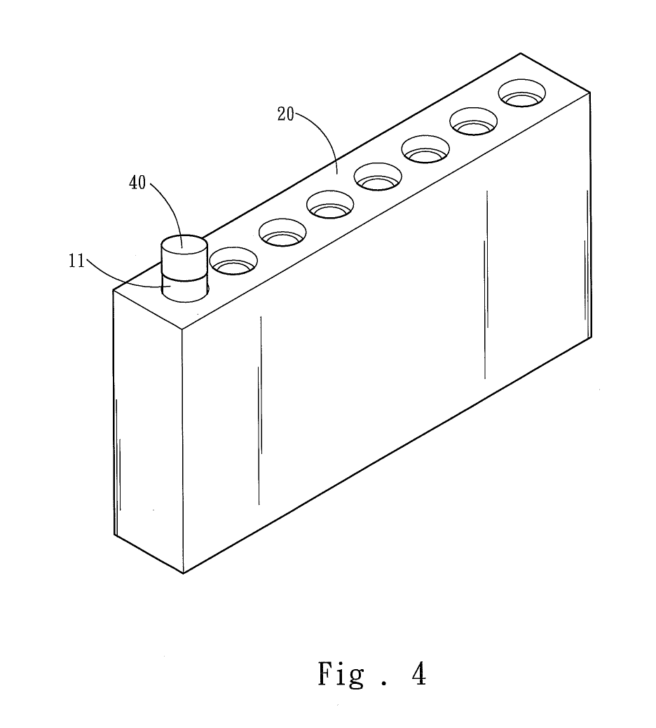

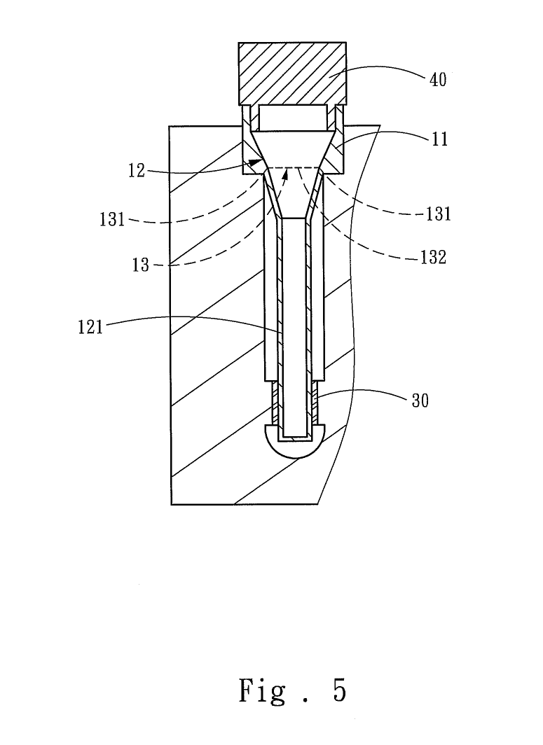

Please refer to FIGS. 3, 4 and 5, the present invention aims to provide a capillary adopted for an apparatus of insulated isothermal polymerase chain reaction that is held on a test tube holder 20 and includes a tube 11 which has an elongated tubular housing space 12. The elongated tubular housing space 12 is cut to form an axial cross section 13 with a distance between left and right edges 131 greater than the distance between front and rear edges 132. The axial cross section 13 can be a closed curved surface with the curvature of the left and right edges 131 greater than that of the front and rear edges 132, thus the axial cross section 13 can be formed in an elliptic shape (as shown in the drawings for discussion herein). The axial cross section 13 also can be formed in a rectangular shape.

The tube 11 can be coupled with a high thermal conductive ring 30 which can be made of metal such as copper at a position close to the bottom of the elongated tubular housing space 12. The tube 11 also can be coupled with a cap 40. The bottom of the elongated tubular housing space 12 can also be gradually shrunk to form an elongated narrow space 121 to improve identification.

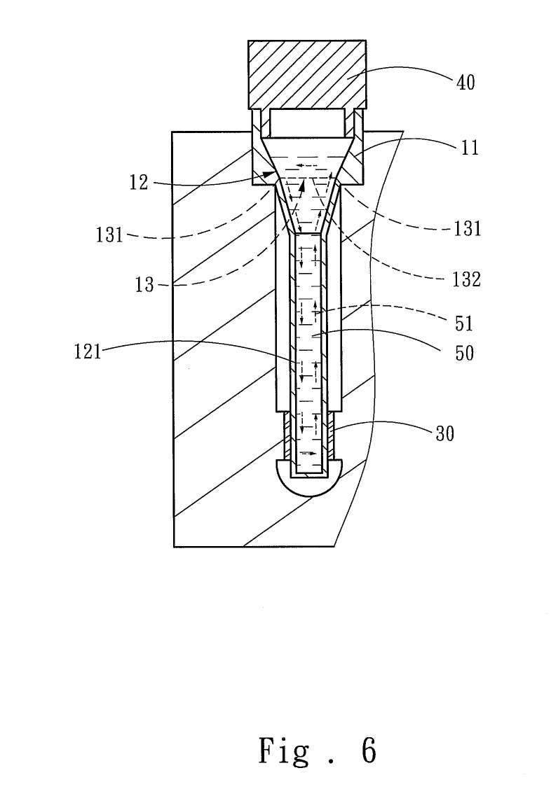

Please refer to FIG. 6, when the reactants 50 are filled in the elongated tubular housing space 12 and heated to generate convection 51, due to the distance between the left and right edges 131 of the axial cross section 13 is greater than that between the front and rear edges 132 and the curvature of the left and right edges 131 and the front and rear edges 132 are different, a narrower passage is formed on the left and right edges 131 of the axial cross section 13, hence can reduce the flowing speed of the heat flow in the convection 51 and increase the cycling duration thereof. As a result, reaction duration of the reactants 50 can be prolonged and reaction efficiency of the apparatus of insulated isothermal polymerase chain reaction also can be increased.

As a conclusion, the present invention provides a non-circular capillary to reduce flowing speed of the convection 51 and increase the reaction duration of the reactants 50, thus can increase the reaction efficiency of the apparatus of insulated isothermal polymerase chain reaction to meet use requirements.

What is claimed is:

1. A capillary for apparatus of insulated isothermal polymerase chain reaction that is held on a test tube holder, comprising:
a tube including an elongated tubular housing space which is cut to form an axial cross section, the axial cross section including left and right edges spaced from each other at a distance greater than that between front and rear edges of the axial cross section.

2. The capillary of claim 1, wherein the axial cross section is a closed curved surface and formed at a curvature of the left and right edges greater that that of the front and rear edges.

3. The capillary of claim 2, wherein the axial cross section is formed in an elliptic shape.

4. The capillary of claim 1, wherein the axial cross section is formed in a rectangular shape.

5. The capillary of claim 1, wherein the tube is coupled with a high thermal conductive ring at a position close to the bottom of the elongated tubular housing space.

6. The capillary of claim 5, wherein the high thermal conductive ring is a metal ring.

7. The capillary of claim 6, wherein the high thermal conductive ring is made of copper.

8. The capillary of claim 1, wherein the tube is coupled with a cap.

9. The capillary of claim 1, wherein the bottom of the elongated tubular housing space is gradually shrunk to form an elongated narrow space.
